(19) United States
(12) Patent Application Publication
(10) Pub. No.: US 2022/0339213 A1
(43) Pub. Date: Oct. 27, 2022

(54) PROBIOTIC COMPOSITIONS AND METHODS OF USE THEREOF

(71) Applicant: Schism Bioworks LLC, Broomfield, CO (US)

(72) Inventors: Sebastian Balcombe, Evergreen, CO (US); Ralf Jäger, Whitefish Bay, WI (US); Martin Purpura, Spring, TX (US)

(21) Appl. No.: 17/730,850

(22) Filed: Apr. 27, 2022

Related U.S. Application Data
(60) Provisional application No. 63/180,520, filed on Apr. 27, 2021.

(51) Int. Cl. A61K 35/741; A61K 35/747; A61K 35/744; A61K 35/742; A61K 36/064; A61K 35/745

(52) U.S. Cl. CPC A61K 35/741; A61K 35/747; A61K 35/744; A61K 35/742; A61K 36/064; A61K 35/745

(57) ABSTRACT

Disclosed herein is method for increasing vitamin B12 levels in a subject comprising administering to the subject a composition comprising: at least one strain of bacteria wherein the at least one strain of bacteria comprises Propionibacterium freudenreichii UF (UF1). In certain embodiments, the UF1 is probiotic. In certain implementations, the administration of the composition increases the subject's serum B12 levels by at least about 10% relative to the subject's serum B12 level prior to administration of the composition. In further embodiments, the administration of the composition confers at least on benefit on the subject selected from; an increase in serum levels of at least one of vitamin B2, B5, B6, B9, and/or B12; an increase in at least one of tryptophan, tyrosine, phenylalanine; increased propionic acid, immune system modulation and/or augmentation, regulation of TH17 cells; and/or reduction in pro-inflammatory metabolites.

### CROSS-REFERENCE TO RELATED APPLICATION(S)

This application claims priority to U.S. Provisional Application No. 63/180,520 filed Apr. 27, 2021, and entitled “PROBIOTIC COMPOSITIONS AND METHODS OF USE THEREOF,” which is hereby incorporated by reference in its entirety under 35 U.S.C. § 119(e).

### BACKGROUND OF THE INVENTION

Vitamin B12 is important for the normal functioning of the brain and nervous system and for the formation of blood. It is involved in the metabolism of every cell of the body, especially affecting the DNA synthesis and regulation but also fatty acid synthesis and energy production. Its effects are still not completely known.

Vitamin B12 deficiencies can result in significant damage to multiple organ systems and supplementation is often required, especially among individuals that have vegetarian or vegan diets, or that are pregnant. There is a need in the art for improved compositions and methods for supplementing vitamin B12.

### BRIEF SUMMARY

Disclosed herein is method for increasing vitamin B12 levels in a subject comprising administering to the subject a composition comprising: at least one strain of bacteria wherein the at least one strain of bacteria comprises Propionibacterium freudenreichii UF (UF1). In certain embodiments, the UF1 is probiotic. In further embodiments, the composition administered delivers a dose of UF1 to the subject from about 5×10⁸ to about 1×10¹¹ colony forming units (CFU). In yet further embodiments, the dose of UF1 is from about 1×10⁹ to about 2×10¹⁰ CFU. in still further embodiments, the dose of UF1 is from about 5×10⁹ to about 1×10¹⁰ CFU. According to certain embodiments, the composition administered delivers a dose of UF1 to the subject from about 5×10⁸ to about 1×10¹¹ Active Fluorescent Units (AFU). In further embodiments, the dose of UF1 is from about 1×10⁹ to about 2×10¹⁰ AFU. In yet further embodiment, the dose of UF1 is from about 5×10⁹ to about 1×10¹⁰ AFU.

In certain embodiments, the UF1 is a mixture of probiotic and paraprobiotic. In exemplary implementations, the UF1 is parabiotic. In further embodiments, the UF1 is a probiotic, parabiotic, prebiotic, synbiotic, or mixture of the foregoing.

In certain implementations, the administration of the composition increases the subject's serum B12 levels by at least about 10% relative to the subject's serum B12 level prior to administration of the composition. In further embodiments, the administration of the composition confers at least on benefit on the subject selected from; an increase in serum levels of at least one of vitamin B2, B5, B6, B9, and/or B12; an increase in at least one of tryptophan, tyrosine, phenylalanine; increased propionic acid, immune system modulation and/or augmentation, regulation of TH17 cells; and/or reduction in pro-inflammatory metabolites. In further embodiments, administration of the composition to the subject increases the level of NAD+ and or NMN levels in the subject.

According to certain implementations, the at least one strain of bacteria comprises at least a second strain of bacteria chosen from: Propionibacterium acnes; Propionibacterium avidum; Propionibacterium granulosum; Propionibacterium lymphophilum; Propionibacterium acidiproprionic; Propionibacterium jensenii Propionibacterium thoenii; lactic acid bacteria including lactobacilli (ssp. acidophilus, fermentum, plantarum, rhamnosus, casei, reuteri, gasseri) and streptococci (e.g. salivarius, thermophilus), spore forming bacteria including Bacillus (e.g. coagulans, subtilis), bifidobacteria, yeast-derived bacteria including Saccharomyces boulardii, and/or mixtures of the foregoing.

According to certain embodiments, the subject is an infant. In exemplary implementations of these embodiments, administration of the composition to the infant accelerates T-cell immunity, decreases neonatal adaptive immunity period, and/or increases pathogen resistance. In certain implementations, the infant was born preterm.

Further disclosed herein is a method of slowing aging in a subject, comprising administering to the subject one or more sirtuin enhancer to the subject, wherein the one or more sirtuin enhancer comprises at least one strain of bacteria wherein the at least one strain of bacteria comprises Propionibacterium freudenreichii UF (UF1).

Further disclosed herein is a probiotic composition comprising Propionibacterium freudenreichii UF (UF1) and at least a second strain of probiotic bacteria wherein the second strain of probiotic bacteria is selected from: Propionibacterium acnes; Propionibacterium avidum; Propionibacterium granulosum; Propionibacterium lymphophilum; Propionibacterium acidiproprionic; Propionibacterium jensenii Propionibacterium thoenii; lactic acid bacteria including lactobacilli (ssp. acidophilus, fermentum, plantarum, rhamnosus, casei, reuteri, gasseri) and streptococci (e.g. salivarius, thermophilus), spore forming bacteria including Bacillus (e.g. coagulans, subtilis), bifidobacteria, yeast-derived bacteria including Saccharomyces boulardii, prebiotics, and synbiotics and/or mixtures of the foregoing.

### DETAILED DESCRIPTION

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

Certain materials, compounds, compositions, and components disclosed herein can be obtained commercially or readily synthesized using techniques generally known to those of skill in the art. For example, the starting materials and reagents used in preparing the disclosed compounds and compositions are either available from commercial suppliers such as Aldrich Chemical Co., (Milwaukee, Wis.), Acros Organics (Morris Plains, N.J.), Fisher Scientific (Pittsburgh, Pa.), or Sigma (St. Louis, Mo.) or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991); March's Advanced Organic Chemistry, (John Wiley and Sons, 4th Edition); and Larock's Comprehensive Organic Transformations (VCH Publishers Inc., 1989).

As used herein, “probiotic bacteria” or “probiotics” as used herein, refers to microorganisms which when administered for nutritional supplementation in adequate amounts confer a health benefit to the subject. Heath benefit can be measured by any method known in the art, for example, as indicated by biochemical markers including for example, blood sugar levels, lipid chemistry, liver function, full blood count, thyroid function, markers of inflammation (ESR and C-reactive protein), and stool classifications.

A probiotic, such as the composition disclosed herein, is optionally combined with at least one suitable prebiotic compound. A prebiotic compound is usually a non-digestible carbohydrate such as an oligo- or polysaccharide, or a sugar alcohol, which is not degraded or absorbed in the upper digestive tract. Known prebiotics include commercial products such as inulin and transgalacto-oligosaccharides.

As used herein, “paraprobiotic bacteria” or “paraprobiotic” means those products comprising killed or inactivated microbes which may positively affect subject health.

As used herein, the term “probiotic composition” can refer to a composition containing at least one species, genus, family, strain, order, or class of probiotic bacteria (e.g., a single isolate or a combination of desired bacteria), and can also include any additional carriers, excipients, and/or therapeutic agents that can be administered to a mammal. In certain embodiments, the probiotic composition comprises a buffering agent to allow the probiotic bacteria to survive in the acidic environment of the stomach, that is, the probiotic bacteria resist low pH and are able to survive passage through the stomach to colonize and grow in the intestinal milieu. Buffering agents can include, for example, sodium bicarbonate, milk, yoghurt, infant formula, and other dairy products. In certain embodiments, the probiotic composition is formulated as a food additive. In certain embodiments, the probiotic composition includes other materials known in the art for inclusion in food additives, such as water or other aqueous solutions, starch, binders, thickeners, colorants, flavorants, odorants, acidulants (e.g., lactic acid or malic acid, among others), vitamins, or minerals, among others.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration. Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of an ingredient or element may still actually contain such item as long as there is no measurable effect thereof.

Propionibacterium freudenreichii is a gram-positive, non-motile bacterium that plays an important role in the creation of Emmental cheese, and to some extent, Jarlsberg cheese, Leerdammer and Maasdam cheese. Its concentration in Swiss-type cheeses is higher than in any other cheese. Propionibacterium strain, P. UF1, has been isolated from the gut microbiota of preterm infants. P. UF1 is also referred to herein as Propionibacterium freudenreichii UF1 or UF1. Disclosed herein is a method for increasing vitamin B12 levels in a subject comprising administering to the subject a composition comprising: at least one strain of bacteria wherein the at least one strain of bacteria comprises Propionibacterium freudenreichii UF (UF1). In certain embodiments, the composition administered delivers a dose of UF1 to the subject from about 5×10⁸ to about 1×10¹¹ colony forming units (CFU). In further embodiments, the dose of UF1 provided is from about 1×10⁹ to about 2×10¹⁰ CFU. In still further embodiments, the dose of UF1 is from about 5×10⁹ to about 1×10¹⁰ CFU.

In certain further embodiments, the amount of UF1 present in a dose is determined by flow cytometry to determine the number of live cells, as measured by Active Fluorescent Units (AFU). In certain aspects, the composition administered delivers a dose of UF1 to the subject from about 5×10⁸ to about 1×10¹¹ AFU. In further embodiments, the dose of UF1 provided is from about 1×10⁹ to about 2×10¹⁰ AFU. In still further embodiments, the dose of UF1 is from about 5×10⁹ to about 1×10¹⁰ AFU.

In certain embodiments, the at least one strain of bacteria is a probiotic. In further embodiments, the at least once strain of bacteria is a paraprobiotic.

In yet further embodiments, the composition further comprises a prebiotic and/or a synbiotic. In even further embodiments, the at least on strain of bacteria is a mixture of a probiotic and paraprobiotic, prebiotic, and/or synbiotic.

According to certain aspects, administration of the disclosed composition increases the subject's serum B12 levels. This is surprising given that it has been viewed as unlikely that administration of a probiotic vB12 producing bacteria would be able to affect serum vB12 levels. This is because while Propionibacterium UF1 is known to produce vB12 under industrial or lab conditions, it is unlikely that a substrate required by make B12 by UF1 occurs in the human gut. Furthermore, B12 is absorbed in the small intestine. B12 must be combined with intrinsic factor, a protein that is produced in the stomach to be absorbed. Probiotics usually reside (they are transient and do not colonize) in the large intestine, meaning the probiotic would be making B12 in the large intestine which is past its site of absorption.

In certain embodiments, administration of the disclosed composition to a subject results in an increases in serum B12 level in the subject by at least 5%, at least 10%, at least 25%, at least 50%, at least 60%, at least 70%, at least 80%, at least 90%, or more, compared to the subject's B12 serum level prior to administration of the composition.

In certain aspects, administration of the disclosed composition confers at least or many benefits on the subject. In certain aspects, the subject has reduced inflammation. In another embodiments, the subject has an increase in serum levels of at least one of vitamin B2, B5, B6, B9, and/or B12. In a further embodiment, the subject has an increase in levels of at least one amino acid. These include, but are not limited to tryptophan, tyrosine, and/or phenylalanine. In a further embodiment, the subject has increased levels of propionic acid, which in turn can has a positive impact on the intestinal regulatory cells, including induced T regulatory cells (Treg cells).

In further embodiments, the subject has benefits to immune system modulation and/or augmentation. In yet further embodiments, the subject has beneficial effects on the regulation of TH17 cells. In still further embodiments, the subject has a reduction in pro-inflammatory metabolites.

According to certain embodiments, the probiotic composition also includes one or more additional strain of bacteria. In certain implementations, this may include one or more of: Propionibacterium acnes; Propionibacterium avidum; Propionibacterium granulosum; Propionibacterium lymphophilum; Propionibacterium acidiproprionic; Propionibacterium jensenii Propionibacterium thoenii; lactic acid bacteria including lactobacilli (ssp. acidophilus, fermentum, plantarum, rhamnosus, casei, reuteri, gasseri) and streptococci (e.g. salivarius, thermophilus), spore forming bacteria including Bacillus (e.g. coagulans, subtilis), bifidobacteria, yeast-derived bacteria including Saccharomyces boulardii, and/or mixtures of the foregoing. In certain further embodiments, second strain of bacteria is a probiotic or a parabiotic or a mixture of the foregoing. In further embodiments, the composition further comprises a prebiotic, and/or synbiotic or mixtures of the foregoing.

According to certain further embodiments, the subject is an infant. In exemplary implementations, the infant experiences one or more of the following benefits from administration of the probiotic composition: accelerated T-cell immunity, decreased neonatal adaptive immunity period, and/or increased pathogen resistance. In certain exemplary implementations, the infant was born preterm.

Further disclosed herein is reducing inflammation in a subject in need thereof, comprising administering to the subject a composition disclosed herein. In certain embodiments the subject has an inflammatory disease or condition. An inflammatory disease or condition may include, but is not limited to: acne vulgaris, asthma, COPD, autoimmune diseases, celiac disease, chronic (plaque) prostatitis, glomerulonephritis, hypersensitivities, inflammatory bowel diseases (IBD, Crohn's disease, ulcerative colitis), pelvic inflammatory disease, reperfusion injury, rheumatoid arthritis, sarcoidosis, transplant rejection, vasculitis, interstitial cystitis, atherosclerosis, allergies (type 1, 2, and 3 hypersensitivity, hay fever), inflammatory myopathies, as systemic sclerosis, and include dermatomyositis, polymyositis, inclusion body myositis, Chediak-Higashi syndrome, chronic granulomatous disease, Vitamin A deficiency, cancer (solid tumor, gallbladder carcinoma), periodontitis, granulomatous inflammation (tuberculosis, leprosy, sarcoidosis, and syphilis), fibrinous inflammation, purulent inflammation, serous inflammation, ulcerative inflammation, and ischemic heart disease, type I diabetes, and diabetic nephropathy. In certain exemplary implementations, the inflammatory disease or condition is Necrotizing enterocolitis. In further exemplary implementations, administration of the probiotic composition reduces prostaglandin E1 and/or 20-hydroxyleukotriene E4 in the subject.

Further disclosed herein is probiotic composition comprising UF1 and at least a second strain of probiotic bacteria and an acceptable carrier thereof. In certain embodiments, the second strain of probiotic bacteria is selected from: Propionibacterium acnes; Propionibacterium avidum; Propionibacterium granulosum; Propionibacterium lymphophilum; Propionibacterium acidiproprionic; Propionibacterium jensenii Propionibacterium thoenii; and/or mixtures of the foregoing. “Acceptable carrier,” as used herein, includes any carrier which does not interfere with the effectiveness of the biological activity of the active ingredients and/or that is not toxic to the subject receiving such ingredients. Non-limiting examples of acceptable carriers include phosphate buffered saline solutions, water, emulsions, such as oil/water emulsions, various types of wetting agents and sterile solutions. Additional non-limiting examples of compatible carriers can include any suitable vehicle, delivery or dispensing means or material. Such acceptable carriers can be formulated by conventional methods and can be administered to the subject at an effective amount.

The disclosed probiotic bacteria according to the invention may be produced using any standard fermentation process known in the art. For example, solid substrate or submerged liquid fermentation. The fermented cultures can be mixed cultures, or single isolates. The probiotic bacteria are anaerobically fermented. The probiotic bacteria are a combination of solid substrate and a submerged liquid fermentation.

The disclosed compositions are formulated for oral administration including chewable foods, beverages, liquids, tablets, capsules, powders, and granulates. In a preferred embodiment the compositions have been formulated into a tablet. In another preferred embodiment the compositions have been formulated into a capsule. In yet another preferred embodiment the compositions have been formulated into granulated or water soluble powders. Further, in certain embodiments, compositions can be formulated into liquids, creams, lotions, gels dispersions or ointments for topical administration.

When formulated the composition may contain further ingredients, including ingredients that have a favorable impact on health, flavor, formulating or tableting. Non-limiting examples of additional ingredients that may suitably be incorporated in the present composition are: vitamins, minerals, nutritional supplements (e.g., fiber), fungal extracts, botanical extracts, sweeteners, flow aids, and fillers.

Tableting aids include for example, carboxylic acids such as malic, maleic, citric, iso-citric and succinic, and salts thereof, SiO₂, Aloe Vera, saturated and unsaturated linear and branched fatty acids and their salts, or fatty alcohols. Preferred tableting aides are malic acid, citric acid, stearic acid or Magnesium stearate. Tableting aides are formulated at a concentration of about 1% to 10%, w/w, 2.5% and 7.5 w/w % or any specific value within said range.

Further disclosed herein is a method of increasing the level of NAD+ and/or NMN levels in a subject comprising administering to the subject a composition comprising at least one strain of bacteria wherein the at least one strain of bacteria comprises Propionibacterium freudenreichii UF (UF1).

Further disclosed herein is a method of slowing aging in a subject, comprising administering to the subject one or more sirtuin enhancer to the subject, wherein the one or more sirtuin enhancer comprises at least one strain of bacteria wherein the at least one strain of bacteria comprises Propionibacterium freudenreichii UF (UF1). In certain embodiments, administration of the sirtuin enhancer to the subject potentiates DNA repair of the subject.

### Nutritional Supplements

The compositions of the disclosure may take the form of dietary supplements or may themselves be used in combination with dietary supplements, also referred to herein as food supplements.

Nutritional supplements may be found in many forms such as tablets, capsules, soft gels, gel caps, liquids, or powders. Some dietary supplements can help ensure an adequate dietary intake of essential nutrients; others may help reduce risk of disease.

The compositions of the disclosure may take the form of a food product. Here, the term “food” is used in a broad sense and covers food and drink for humans as well as food and drink for animals (i.e. a feed). Preferably, the food product is suitable for, and designed for, human consumption.

The food may be in the form of a liquid, solid or suspension, depending on the use and/or the mode of application and/or the mode of administration.

When in the form of a food product, the composition may comprise or be used in conjunction with one or more of: a nutritionally acceptable carrier, a nutritionally acceptable diluent, a nutritionally acceptable excipient, a nutritionally acceptable adjuvant, a nutritionally active ingredient.

By way of example, the compositions of the disclosure may take the form of one of the following: A fruit juice; a beverage comprising whey protein: a health or herbal tea, a cocoa drink, a coffee drink, a yoghurt and/or a drinking yoghurt, a cheese, an ice cream, a desserts, a confectionery, a biscuit, a cake, cake mix or cake filling, a snack food, a fruit filling, a cake or doughnut icing, an instant bakery filling cream, a filling for cookies, a ready-to-use bakery filling, a reduced calorie filling, an adult nutritional beverage, an acidified soy/juice beverage, a nutritional or health bar, a beverage powder, a calcium fortified soy milk, or a calcium fortified coffee beverage.

Compositions of the present disclosure may take the form of a food ingredient and/or feed ingredient. As used herein the term “food ingredient” or “feed ingredient” includes a composition which is or can be added to functional foods or foodstuffs as a nutritional and/or health supplement for humans and animals.

The food ingredient may be in the form of a liquid, suspension or solid, depending on the use and/or the mode of application and/or the mode of administration.

Compositions of the disclosure may take the form of functional foods.

As used herein, the term “functional food” means food which is capable of providing not only a nutritional effect, but is also capable of delivering a further beneficial effect to the consumer.

Various aspects and embodiments of the present invention are defined by the following numbered clauses:

1. A probiotic composition comprising Propionibacterium freudenreichii UF (UF1).
2. The composition of clause 1, further comprising at least a second strain of probiotic bacteria.
3. The probiotic composition of clause 2, wherein the second strain of probiotic bacteria is selected from: Propionibacterium acnes; Propionibacterium avidum; Propionibacterium granulosum; Propionibacterium lymphophilum; Propionibacterium acidiproprionic; Propionibacterium jensenii Propionibacterium thoenii; lactic acid bacteria including lactobacilli (ssp. acidophilus, fermentum, plantarum, rhamnosus, casei, reuteri, gasseri) and streptococci (e.g. salivarius, thermophilus), spore forming bacteria including Bacillus (e.g. coagulans, subtilis), bifidobacteria, yeast-derived bacteria including Saccharomyces boulardii, prebiotics, and synbiotics and/or mixtures of the foregoing.
4. The probiotic composition any of clauses 1-3, further comprising a probiotic stabilizer.
5. The probiotic composition of clause 4, wherein the probiotic stabilizer is maltodextrin.
6. A method for increasing vitamin B12 levels in a subject comprising administering to the subject a composition the probiotic composition of any of clauses 1-10.
7. The method of clause 1, wherein the UF1 is probiotic.
8. The method of clause 7, wherein the composition administered delivers a dose of UF1 to the subject from about 5×10⁸ to about 1×10¹¹ colony forming units (CFU).
9. The method of clause 8, wherein the dose of UF1 is from about 1×10⁹ to about 2×10¹⁰ CFU.]
10. The method of clause 9, wherein the dose of UF1 is from about 5×10⁹ to about 1×10¹⁰ CFU.
11. The method of clause 6, wherein the composition administered delivers a dose of UF1 to the subject from about 5×10⁸ to about 1×10¹¹ Active Fluorescent Units (AFU).
12. The method of clause 11, wherein the dose of UF1 is from about 1×10⁹ to about 2×10¹⁰ AFU.
13. The method of clause 12, wherein the dose of UF1 is from about 5×10⁹ to about 1×10¹⁰ AFU.
14. The method of clause 6, wherein the UF1 is a mixture of probiotic and paraprobiotic.
15. The method of clause 6, wherein the UF1 is parabiotic.
16. The method of clause 15, wherein the parabiotic UF1 is inactivated through heat treatment, or chemicals, or gamma rays, or ultraviolet rays, or sonication.
17. The method of clause 6, wherein the UF1 is a probiotic, parabiotic, prebiotic, synbiotic, or mixture of the foregoing.
18. The method of any preceding clause, wherein the administration of the composition increases the subject's serum B12 levels.
19. The method of any preceding clause, wherein the administration of the composition confers at least on benefit on the subject selected from; an increase in serum levels of at least one of vitamin B2, B5, B6, B9, and/or B12; an increase in at least one of tryptophan, tyrosine, phenylalanine; increased propionic acid, immune system modulation and/or augmentation, regulation of TH17 cells; and/or reduction in pro-inflammatory metabolites.
20. The method of any preceding clause, wherein the at least one strain of bacteria comprises at least a second strain of bacteria.
21. The method of clause 20, wherein the second strain of bacteria is chosen from: Propionibacterium acnes; Propionibacterium avidum; Propionibacterium granulosum; Propionibacterium lymphophilum; Propionibacterium acidiproprionic; Propionibacterium jensenii Propionibacterium thoenii; lactic acid bacteria including lactobacilli (ssp. acidophilus, fermentum, plantarum, rhamnosus, casei, reuteri, gasseri) and streptococci (e.g. salivarius, thermophilus), spore forming bacteria including Bacillus (e.g. coagulans, subtilis), bifidobacteria, yeast-derived bacteria including Saccharomyces boulardii, and/or mixtures of the foregoing.
22. The method of clause 21 wherein the second strain of bacteria is a probiotic, parabiotic, prebiotic, and/or synbiotic, and/or mixture of the foregoing.
23. The method of any preceding clause, wherein the subject is an infant.
24. The method of clause 23, wherein administration of the composition to the infant accelerates T-cell immunity, decreases neonatal adaptive immunity period, and/or increases pathogen resistance.
25. The method of clause 24, wherein the infant was born preterm.
26. A method for increasing vitamin B12 levels in a subject comprising administering to the subject a composition comprising: at least one species of bacteria wherein the at least species of bacteria comprises the species Propionibacterium freudenreichii (PF)
27. The method of clause 26, wherein the composition administered delivers a dose of PF to the subject from about 5×10⁸ to about 1×10¹¹ CFU.
28. The method of clause 27, wherein the dose of PF is from about 1×10⁹ to about 2×10¹⁰ CFU.
29. The method of clause 28, wherein the dose of PF is from about 5×10⁹ to about 1×10¹¹ CFU.
30. The method of clause 26, wherein the composition administered delivers a dose of PF to the subject from about 5×10⁸ to about 1×10¹¹ AFU.
31. The method of clause 30, wherein the dose of PF is from about 1×10⁹ to about 2×10¹⁰ AFU.
32. The method of clause 31, wherein the dose of PF is from about 5×10⁹ to about 1×10¹⁰.
33. The method of clause any preceding clause, wherein the composition further comprises a probiotic stabilizer (e.g. maltodextrin).
34. A method of increasing the level of NAD+ and or NMN levels in a subject comprising administering to the subject a composition comprising: at least one strain of bacteria wherein the at least one strain of bacteria comprises Propionibacterium freudenreichii UF (UF1).
35. A method of slowing aging in a subject, comprising administering to the subject one or more sirtuin enhancer to the subject, wherein the one or more sirtuin enhancer comprises at least one strain of bacteria wherein the at least one strain of bacteria comprises Propionibacterium freudenreichii UF (UF1).
36. The method of clause 35, wherein administration of the sirtuin enhancer to the subject potentiates DNA repair of the subject.

While multiple embodiments are disclosed, still other embodiments of the disclosure will become apparent to those skilled in the art from the following detailed description, which shows and describes illustrative embodiments of the disclosed compositions, systems and methods. As will be realized, the disclosed compositions, systems and methods are capable of modifications in various obvious aspects, all without departing from the spirit and scope of the disclosure. Accordingly, the detailed description is to be regarded as illustrative in nature and not restrictive.

What is claimed is:

1. A method for increasing vitamin B12 levels in a subject comprising administering to the subject a composition comprising: at least one strain of bacteria wherein the at least one strain of bacteria comprises Propionibacterium freudenreichii UF (UF1).

2. The method of claim 1, wherein the UF1 is probiotic.

3. The method of claim 2, wherein the composition administered delivers a dose of UF1 to the subject from about 5×10⁸ to about 1×10¹¹ colony forming units (CFU).

4. The method of claim 3, wherein the dose of UF1 is from about 1×10⁹ to about 2×10¹⁰ CFU.

5. The method of claim 4, wherein the dose of UF1 is from about 5×10⁹ to about 1×10¹⁰ CFU.

6. The method of claim 1, wherein the composition administered delivers a dose of UF1 to the subject from about 5×10⁸ to about 1×10¹¹ Active Fluorescent Units (AFU).

7. The method of claim 6, wherein the dose of UF1 is from about 1×10⁹ to about 2×10¹⁰ AFU.

8. The method of claim 7, wherein the dose of UF1 is from about 5×10⁹ to about 1×10¹⁰ AFU.

9. The method of claim 1, wherein the UF1 is a mixture of probiotic and paraprobiotic.

10. The method of claim 1, wherein the UF1 is parabiotic.

11. The method of claim 1, wherein the UF1 is a probiotic, parabiotic, prebiotic, synbiotic, or mixture of the foregoing.

12. The method of claim 1, wherein the administration of the composition increases the subject's serum B12 levels by at least about 10% relative to the subject's serum B12 level prior to administration of the composition.

13. The method of claim 1, wherein the administration of the composition confers at least on benefit on the subject selected from; an increase in serum levels of at least one of vitamin B2, B5, B6, B9, and/or B12; an increase in at least one of tryptophan, tyrosine, phenylalanine; increased propionic acid, immune system modulation and/or augmentation, regulation of TH17 cells; and/or reduction in pro-inflammatory metabolites.

14. The method of claim 1, wherein administration of the composition to the subject increases the level of NAD+ and or NMN levels in the subject.

15. The method of claim 1, wherein the at least one strain of bacteria comprises at least a second strain of bacteria chosen from: Propionibacterium acnes; Propionibacterium avidum; Propionibacterium granulosum; Propionibacterium lymphophilum; Propionibacterium acidiproprionic; Propionibacterium jensenii Propionibacterium thoenii; lactic acid bacteria including lactobacilli (ssp. acidophilus, fermentum, plantarum, rhamnosus, casei, reuteri, gasseri) and streptococci (e.g. salivarius, thermophilus), spore forming bacteria including Bacillus (e.g. coagulans, subtilis), bifidobacteria, yeast-derived bacteria including Saccharomyces boulardii, and/or mixtures of the foregoing.

16. The method of claim 1, wherein the subject is an infant.

17. The method of claim 16, wherein administration of the composition to the infant accelerates T-cell immunity, decreases neonatal adaptive immunity period, and/or increases pathogen resistance.

18. The method of claim 17, wherein the infant was born preterm.

19. A method of slowing aging in a subject, comprising administering to the subject one or more sirtuin enhancer to the subject, wherein the one or more sirtuin enhancer comprises at least one strain of bacteria wherein the at least one strain of bacteria comprises Propionibacterium freudenreichii UF (UF1).

20. A probiotic composition comprising Propionibacterium freudenreichii UF (UF1) and at least a second strain of probiotic bacteria wherein the second strain of probiotic bacteria is selected from: Propionibacterium, acnes; Propionibacterium avidum; Propionibacterium granulosum; Propionibacterium lymphophilum; Propionibacterium acidiproprionic; Propionibacterium jensenii Propionibacterium thoenii; lactic acid bacteria including lactobacilli (ssp. acidophilus, fermentum, plantarum, rhamnosus, casei, reuteri, gasseri) and streptococci (e.g. salivarius, thermophilus), spore forming bacteria including Bacillus (e.g. coagulans, subtilis), bifidobacteria, yeast-derived bacteria including Saccharomyces boulardii, prebiotics, and synbiotics and/or mixtures of the foregoing.
